(19) United States
(12) Patent Application Publication
(10) Pub. No.: US 2007/0282297 A1
(43) Pub. Date: Dec. 6, 2007

(54) System and method for safely infusing toxins using extension set, connect set and cyto admin set

(76) Inventor: Thomas F. Knight, Trabuco Canyon, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 11/446,447

(22) Filed: Jun. 1, 2006

(51) Int. Cl. A61M 31/00

(52) U.S. Cl. 604/500

(57) ABSTRACT

The present invention provides a method for safely infusing a toxic fluid into a patient using an extension set, a connect set and a cyto admin set, the extension set comprising an extension set line, and first and second syringes, wherein the extension set line provides fluid communication between a toxic fluid container and the syringes such that toxic fluid may be moved from within the toxic fluid container into one of the syringes, the connect set comprising a connect set line in fluid communication with an infusion bag, wherein the connect set includes a port for connection with the extension set such that the syringes are in fluid communication with the infusion bag, the cyto admin set comprising one or more check valves for connection with the connect set such that the infusion bag is in fluid communication with the cyto admin set, the method comprising drawing the toxic fluid from the toxic fluid container through the extension set line into the first syringe, flushing the extension set line using fluid from the second syringe, disconnecting the extension set from the toxic fluid container, attaching the extension set to the connect set such that fluid communication is established therebetween, injecting the toxic fluid from the first syringe into the infusion bag, flushing the extension set line using fluid from the second syringe, attaching the connect set to the cyto admin set and infusing the toxic fluid from the infusion bag into the patient.

### FIELD OF THE INVENTION

The invention broadly relates to a system and method for safely infusing toxins and more particularly to a system and method for enabling toxic solutions such as used in chemotherapy to be safely infused into a patient using an extension set, a connect set and a cyto admin set with a significantly reduced risk of contacting the patient or the handler(s) performing the infusion

### BACKGROUND OF THE INVENTION

Many medical applications involve the infusion of dangerous medicines such as toxic solutions, radioactive fluid or other dangerous fluid into a patient's body. These dangerous medicines include chemotherapy, biologically active substances, radiosensitizers and cytotoxics, which may be infused directly into a patient through an intravenous tube. Other medical applications involve infusing the fluid to a medical device located within or in proximity to the patient. In the case of toxic fluid syringe injections, current methods of syringe shielding may not provide the patient or the handler with the total protection needed in terms of radiation shielding or containment of a spill or leak of the infusion system.

In a typical application, toxic fluid are supplied to a patient via a delivery system, and then the delivery system is flushed with saline or some other non-hazardous dilutant. The infusion, removal and flushing of the radioactive fluid often results in the use of several syringes and fluid lines that must be interchanged in the infusion system with a resulting increase in the possibility that toxic fluid will leak or spill, thereby contaminating the surrounding environment. Syringe shields are currently available and are generally made to shield a syringe filled with toxic fluid by employing lead as a means of shielding against toxic radioactivity. However, these devices protect patients and handlers from radioactive fluid rather than containment of potential leaks or spills of toxic fluid.

In view of the above, there exists a need for a system and method for enabling toxic solutions such as used in chemotherapy to be safely infused with a significantly reduced risk of contacting the handler(s) performing the infusion.

### SUMMARY OF THE INVENTION

The present invention provides a system and method for enabling toxic solutions such as used in chemotherapy to be safely infused with a significantly reduced risk of contacting the handler(s) performing the infusion. According to a preferred implementation of the invention, a flush processing apparatus is employed for transferring, mixing and delivering hazardous fluid such as drugs or toxins from a supplied drug container to an intravenous bag or delivery system, wherein the resultant exposure of the drug to the surrounding environment is reduced or eliminated.

One aspect of the invention involves a method for safely infusing a toxic fluid into a patient using an infusion system including an extension set, a connect set and a cyto admin set, the extension set comprising an extension set line, and first and second syringes, wherein the extension set line provides fluid communication between a toxic fluid container and the syringes such that toxic fluid may be moved from within the toxic fluid container into one of the syringes, the connect set comprising a connect set line in fluid communication with an infusion bag, wherein the connect set includes a port for connection with the extension set such that the syringes are in fluid communication with the infusion bag, the cyto admin set comprising one or more check valves for connection with the connect set such that the infusion bag is in fluid communication with the cyto admin set, the method comprising drawing the toxic fluid from the toxic fluid container through the extension set line into the first syringe, flushing the extension set line using fluid from the second syringe, disconnecting the extension set from the toxic fluid container, attaching the extension set to the connect set such that fluid communication is established therebetween, injecting the toxic fluid from the first syringe into the infusion bag, flushing the extension set line using fluid from the second syringe, attaching the connect set to the cyto admin set and infusing the toxic fluid from the infusion bag into the patient.

The method may further comprise the step puncturing the toxic fluid container using a vial spike of the extension set to allow fluid flow of the toxic drug from the toxic fluid container into the first syringe. The vial spike preferably includes an inline filtration membrane that allows the drug to be filtered as it is withdrawn from drug container. A further step includes manually kneading the infusion bag to mix the fluid contained therein before infusing the fluid into the patient. In the method, the step of drawing the toxic fluid into a first syringe comprises turning an extension set stopcock to a first position to allow fluid flow of the toxic fluid from the toxic fluid container, through an extension set line, and into the first extension set syringe. Also, the step of flushing the extension set line using a second syringe includes the step of turning the stopcock to a second position to allow fluid flow between the second syringe and the toxic fluid container such that the second syringe empties approximately one half of a flush solution through the extension set line and into the toxic fluid container.

The stopcock preferably includes a stop post that prevents the flush syringe and the drug syringe from being opened at the same time, which would otherwise compromise the flushing and the fluid barrier protection afforded the handler. The cyto admin set may further comprise a dedicated universal spike adapter for connection with any spike on any conventional pump set. By way of example, the pump set may comprise an administration set having a spike with chamber, a pump key, a length of tubing, a needleless y-site, a roller clamp, and a luer lock for connection with an IV catheter that connects to the patient's blood vessel. In this manner, the dedicated universal spike adapter of the present invention is compatible with existing pump equipment, pump sets, and current hospital protocols such that the connect set will connect to existing pump sets in use with current pumps without affecting the overall function of the device.

Another aspect of the invention involves a system for safely infusing a toxic fluid into a patient, the system comprising: (1) extension set comprising an extension set line, and first and second syringes, the extension set line providing fluid communication between a toxic fluid container and the syringes such that toxic fluid may be moved from within the toxic fluid container into one of the syringes; (2) a connect set comprising a connect set line in fluid communication with an infusion bag, the connect set including a port for connection with the extension set such that the syringes are in fluid communication with the infusion bag; and (3) a cyto admin set comprising one or more check valves for connection with the connect set such that the infusion bag is in fluid communication with the cyto admin set.

In operation, toxic fluid is drawn from the toxic fluid container through the extension set line into the first syringe, and then the extension set line is flushed using fluid from the second syringe. The extension set is disconnected from the toxic fluid container and attached to the connect set such that fluid communication is established therebetween, wherein the toxic fluid from the first syringe is injected into the infusion bag. The extension set line is then flushed using fluid from the second syringe, and the extension set is optionally disconnected and disposed. The connect set is then connected to the cyto admin set, such that the toxic fluid in the infusion bag may be infused into the patient. The cyto admin set may further comprise a dedicated universal spike adapter for connection with any spike on any conventional pump set, such as including a spike, a pump key, a length of tubing, a needleless y-site, a roller clamp, and a luer lock for attachment with an IV catheter that connects to the patient's blood vessel. Because the dedicated universal spike adapter is compatible with existing pump equipment, the connect set may be attached to existing pump sets with current pumps without affecting the overall function of the device.

The extension set may further comprise a vial spike for puncturing the toxic fluid container to allow fluid flow of the toxic drug from the toxic fluid container into the first syringe, wherein the vial spike includes an inline filtration membrane that allows the drug to be filtered as it is withdrawn from drug container. In addition, the infusion bag may be manually kneaded to mix the fluid contained therein before infusing the fluid into the patient. The toxic fluid is drawn into the first syringe by turning an extension set stopcock to a first position to allow fluid flow of the toxic fluid from the toxic fluid container, through an extension set line, and into the first extension set syringe. The extension set line is flushed by turning the stopcock to a second position to allow fluid flow between the second syringe and the toxic fluid container such that the second syringe empties a flush solution through the extension set line and into the toxic fluid container. The stopcock may include a stop post that prevents the flush syringe and the drug syringe from being opened at the same time, which would otherwise compromise the flushing and the fluid barrier protection afforded the handler.

### BRIEF DESCRIPTION OF THE DRAWINGS

FIG. 1 is a schematic view of an extension set of a system for safely infusing a toxic fluid into a patient, in accordance with the principles of the present invention;

FIG. 2 is a schematic view of a the extension set of FIG. 1 attached to a connect set of the system for safely infusing a toxic fluid into a patient, in accordance with the principles of the present invention; and

FIG. 3 is a schematic view of the connect set of FIG. 2 attached to a cyto admin set of the system for enabling toxic solutions to be safely infused into a patient, in accordance with the principles of the present invention.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

The present invention provides a system and method for enabling toxic solutions such as used in chemotherapy to be safely infused into a patient with a considerably lower risk of contacting the patient or the handler(s) performing the infusion. In particular, an infusion and flushing system is used to transfer, mix and deliver hazardous fluid such as drugs or toxins from a supplied drug container to an intravenous bag or delivery system, wherein the drugs or toxins are then infused into the patient.

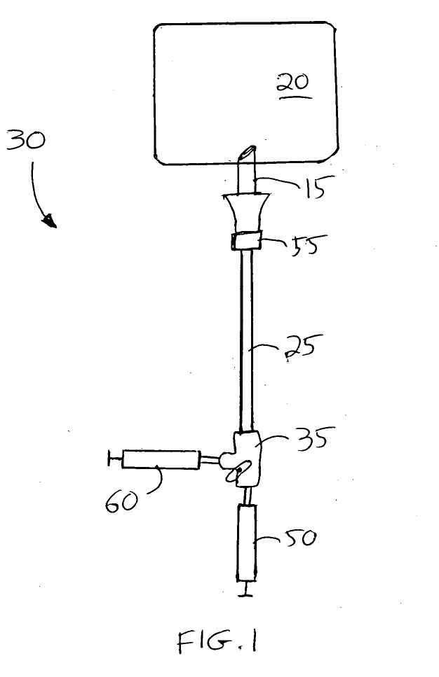

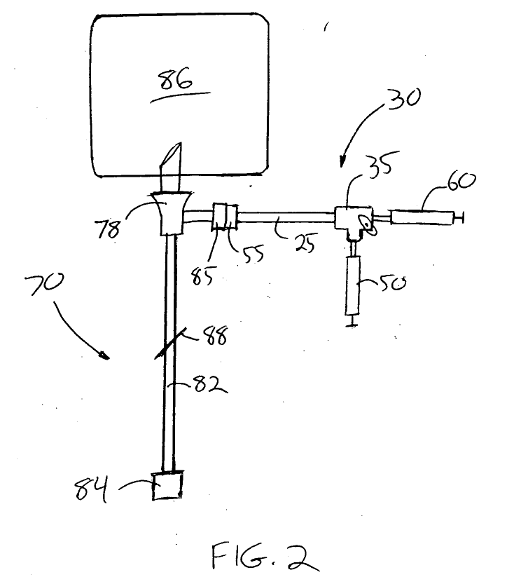

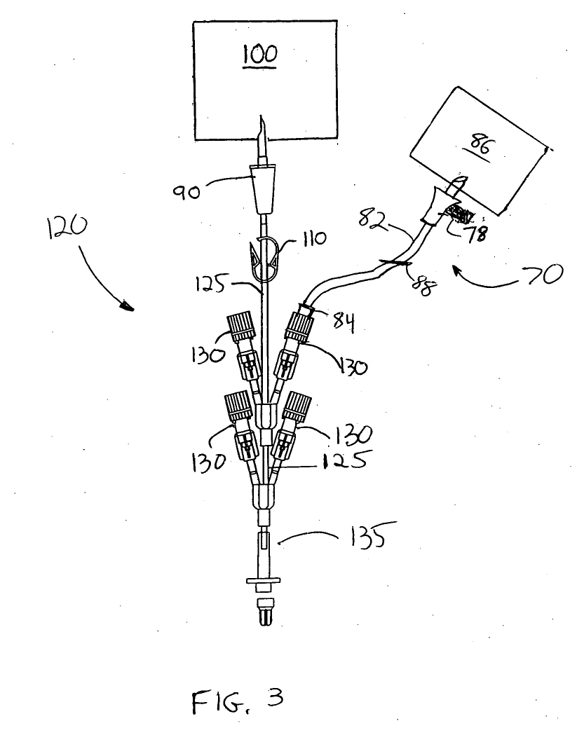

Referring to FIG. 1-3, a preferred system for enabling toxic solutions such as used in chemotherapy to be safely infused with a significantly reduced risk of contacting the patient or the handler(s) performing the infusion will now be described. Specifically, the system comprises an extension set 30 (depicted in FIGS. 1 and 2), a connect set 70 (depicted in FIGS. 2 and 3), and a cyto admin set 120 (depicted in FIG. 3). As illustrated in FIG. 1, the extension set 30 comprises a vial spike 15 for penetrating a drug container 20 in order to withdraw the fluid therefrom, an extension set line 25 (including a stopcock 35) for connecting the vial spike 15 to a pair of extension set syringes 50, 60. The extension set syringes 50, 60 may comprise standard luer locking syringes including a first syringe 50, which is initially empty, and a second syringe 60, which is initially filled with a flush solution. The extension set 30 is attached to the vial spike 15 via a male luer lock 55, which is generally disposed at a distal end of the line 25. The vial spike 15 preferably includes an inline filtration membrane that allows the drug to be filtered as it is withdrawn from the drug container 20.

The stopcock 35 includes three distinct positions comprising a first position which allows fluid flow of the toxic drug from the drug container 20 to the first syringe 50, a second position which allows fluid flow between the second syringe 60 and the drug container 20, and a third position in which all fluid flow is prevented. In accordance with the principles of the invention, the stopcock 35 includes an engineered controlled safety feature in that the stop post on the stopcock 35 prevents the flush syringe 60 and the drug syringe 50 from being opened at the same time, which would otherwise compromise the flushing and the fluid barrier protection afforded the handler. Additionally, the stopcock 35 permits the extension set line 25 to be flushed after drug delivery to rinse out any hazardous drug residue to prevent undue exposure of the toxic drug to the surrounding environment. After withdrawing the toxic fluid from the drug container into the first syringe 50, the stopcock 35 is turned to the second position, whereby the extension set line 25 is flushed by injecting a flush solution from the second syringe 60 through the extension set line 25 and into the drug container 20. At this time, the extension set 30 may be safely detached from the drug container 20.

Turning now to FIG. 2, the connect set 70 comprises a one-way valve port 78 for connecting the connect set line 82 at one end to an infusion bag 86 or other delivery system. The connect set 70 further comprises a connect set line 82, a clamp 88, and a spin lock 84 attached to the other end of the connect set line 82. As illustrated in FIG. 2, the extension set 30 is configured to be attached to the connect set 70 by way of the male luer lock 55, which is attached in a conventional manner to a side port 85 of the one-way valve port 78 to allow the fluids in the syringes 50, 60 to be moved into the infusion bag 86. During this process, the clamp 88 is used to prevent any fluids from entering the connect set line 82. In operation, the toxic fluid located in the first syringe 50 is injected through the extension set line 25 and one-way valve port 78 into the infusion bag 86. Then, the remaining flush solution within the second syringe 60 is injected into the infusion bag 86 in order to flush the extension set line 25 after drug delivery to rinse out any hazardous drug residue and prevent undue exposure of the toxic drug to the surrounding environment. The handler may manually knead the infusion bag 86 to mix the toxic drug and flush solution. At this point, the male luer lock 55 optionally may be disconnected from the side port 85 such that the extension set 30 may be disposed.

Referring to FIG. 3, the cyto admin set 120 comprises a one-way valve port 90 in fluid communication with an intravenous bag 100, a clamp 110, and a primary line 125 for the passage of saline or neutral fluids into the patient. The clamp 110 is used to obstruct the primary line 125 while the toxic fluid is being infused into the patient. The cyto admin set 120 further comprises a plurality of check valves 130 and a dedicated universal spike adapter 135. In the illustrated embodiment, the cyto admin set 120 contains four check valves 130. However, as would be appreciated by those of skill in the art, the cyto admin set 120 may include any number of check valves 130 without departing from the scope of the present invention. The connect set 70 is attached to one of the check valves 130 via the spin lock 84 to permit fluid communication between the connect set 70 and the cyto admin set 120. By way of example, the other check valves 130 may be employed to selectively infuse other solutions into the patient on an as needed basis.

Once fluid communication is established, the toxic fluids within the infusion bag are infused into the patient by way of the connect set line 82, the check valve 130 and the dedicated universal spike adapter 135, which is configured to be connected with virtually any conventional spike on any pump set. The pump set, for example, may comprise a spike, a pump key, a length of tubing, a needleless y-site, a roller clamp, and a luer lock for attachment with an IV catheter that connects to the patient's blood vessel. Since the dedicated universal spike adapter 135 is compatible with existing pump equipment, the connect set 70 may be attached to the existing pump sets without affecting the overall function of the device.

With further reference to FIGS. 1-3, a method of safely infusing toxins into a patient according to the principles of the present invention will now be described. Referring to FIG. 1, the method initially involves puncturing the drug container 20 with the vial spike 15 of the extension set 30 and turning the stopcock 35 to the first position to allow fluid flow of the toxic drug from the drug container 20 to the first syringe 50. In particular, the first syringe 50 is employed to draw fluid from the drug container 20 through the extension set line 25 and into the first syringe 50. The next step involves turning the stopcock 35 to the second position to allow fluid flow between the second syringe 60 and the drug container 20, whereby the second syringe 60 empties approximately one half of the flush solution through the extension set line 25 and into the drug container 20. The subsequent steps entail turning the stopcock 35 to the third, closed position and then disconnecting and capping the male luer lock 55 such the extension set 30 may be handled without fear of exposure to toxic fluid.

Referring to FIG. 2, the next steps involve attaching the male luer lock 55 of the extension set 30 to the side port 85 of the one-way valve port 78 of the connect set 70 to allow the fluids in the syringes 50, 60 to be moved into the infusion bag 86. Another step involves using the clamp 88 to prevent any fluids from entering the connect set line 82. The subsequent step comprises injecting the toxic fluid located in the first syringe 50 into the infusion bag 86 by way of the extension set line 25 and one-way valve port 78. In the next step, the remaining flush solution within the second syringe 60 is injected into the infusion bag 86 in order to flush the extension set line 25 and one-way valve port 78 after drug delivery. Optionally, the male luer lock 55 may be disconnected from the side port 85 and the extension set 30 disposed. In the next step, the handler may manually knead the infusion bag 86 to mix the toxic drug and flush solution.

Referring to FIG. 3, the next steps involve moving the connect set 70 to the vicinity of the patient and then attaching the connect set to the cyto admin set 120. In particular, the connect set 70 is attached to one of the check valves 130 of the cyto admin set 120 via the spin lock 84 to permit fluid communication between the connect set 70 and the cyto admin set 120. The primary line 125 of the cyto admin set 120 for the passage of saline or neutral fluids into the patient may be clamped off (using clamp 110) while the toxic fluid is being infused into the patient. The subsequent steps entail removing the clamp 88 from the connect set line 82 to allow fluid flow from the infusion bag 86 to the patient and infusing the toxic fluid into the patient. After infusion of the toxic fluid, the clamp 1 10 may be removed from the primary line 125 which that the infusion of fluids from the intravenous bag 100 may be resumed.

Thus, it is seen that a system and method for safely infusing toxins is provided. One skilled in the art will appreciate that the present invention can be practiced by other than the various embodiments and preferred embodiments, which are presented in this description for purposes of illustration and not of limitation, and the present invention is limited only by the claims that follow. It is noted that equivalents for the particular embodiments discussed in this description may practice the invention as well.

1. A method for safely infusing a toxic fluid into a patient using an extension set, a connect set and a cyto admin set, the extension set comprising an extension set line, and first and second syringes, wherein the extension set line provides fluid communication between a toxic fluid container and the syringes such that toxic fluid may be moved from within the toxic fluid container into one of the syringes, the connect set comprising a connect set line in fluid communication with an infusion bag, wherein the connect set includes a port for connection with the extension set such that the syringes are in fluid communication with the infusion bag, the cyto admin set comprising one or more check valves for connection with the connect set such that the infusion bag is in fluid communication with the cyto admin set, the method comprising the steps of:
drawing the toxic fluid from the toxic fluid container through the extension set line into the first syringe;
flushing the extension set line using fluid from the second syringe;
disconnecting the extension set from the toxic fluid container;
attaching the extension set to the connect set such that fluid communication is established therebetween;
injecting the toxic fluid from the first syringe into the infusion bag;
flushing the extension set line using fluid from the second syringe;
attaching the connect set to the cyto admin set; and
infusing the toxic fluid from the infusion bag into the patient.

2. The method of claim 1, further comprising the step of puncturing the toxic fluid container using a vial spike of the extension set to allow fluid flow of the toxic drug from the toxic fluid container into the first syringe.

3. The method of claim 2, wherein the vial spike includes an inline filtration membrane that allows the drug to be filtered as it is withdrawn from drug container.

4. The method of claim 1, further comprising the step of clamping off a primary line of the cyto admin set for the passage of saline or neutral fluids into the patient while the toxic fluid is being infused into the patient.

5. The method of claim 1, further comprising the step of manually kneading the infusion bag to mix the fluid contained therein before infusing the fluid into the patient.

6. The method of claim 1, wherein the step of drawing the toxic fluid into a first syringe comprises turning an extension set stopcock to a first position to allow fluid flow of the toxic fluid from the toxic fluid container, through an extension set line, and into the first extension set syringe.

7. The method of claim 6, wherein the step of flushing the extension set line using a second syringe includes the step of turning the stopcock to a second position to allow fluid flow between the second syringe and the toxic fluid container such that the second syringe empties approximately one half of a flush solution through the extension set line and into the toxic fluid container.

8. The method of claim 7, further comprising the step of turning the stopcock to third, closed position prior to disconnecting the extension set.

9. The method of claim 1, wherein the cyto admin set further comprises a dedicated universal spike adapter for connection with any spike on any conventional pump set.

10. The method of claim 1, wherein the stopcock includes a stop post that prevents the first and second syringes from being opened at the same time.

11. A system for safely infusing a toxic fluid into a patient, the system comprising:
an extension set comprising an extension set line, and first and second syringes, the extension set line providing fluid communication between a toxic fluid container and the syringes such that toxic fluid may be moved from within the toxic fluid container into one of the syringes;
a connect set comprising a connect set line in fluid communication with an infusion bag, the connect set including a port for connection with the extension set such that the syringes are in fluid communication with the infusion bag; and
a cyto admin set comprising one or more check valves for connection with the connect set such that the infusion bag is in fluid communication with the cyto admin set;
wherein toxic fluid is drawn from the toxic fluid container through the extension set line into the first syringe;
wherein the extension set line is flushed using fluid from the second syringe;
wherein the extension set is disconnected from the toxic fluid container and attached to the connect set such that fluid communication is established therebetween;
wherein the toxic fluid from the first syringe is injected into the infusion bag;
wherein the extension set line is flushed using fluid from the second syringe;
wherein the connect set is connected to the cyto admin set;
wherein the toxic fluid in the infusion bag is infused into the patient.

12. The system of claim 11, wherein the extension set further comprises a vial spike for puncturing the toxic fluid container to allow fluid flow of the toxic drug from the toxic fluid container into the first syringe.

13. The system of claim 12, wherein the vial spike includes an inline filtration membrane that allows the drug to be filtered as it is withdrawn from drug container.

14. The system of claim 1 1, wherein a primary line of the cyto admin set for the passage of saline or neutral fluids into the patient is clamped off while the toxic fluid is being infused into the patient.

15. The system of claim 11, wherein the infusion bag is manually kneaded to mix the fluid contained therein before infusing the fluid into the patient.

16. The system of claim 1 1, wherein the toxic fluid is drawn into the first syringe by turning a stopcock to a first position to allow fluid flow of the toxic fluid from the toxic fluid container, through an extension set line, and into the first extension set syringe.

17. The system of claim 16, the extension set line is flushed by turning the stopcock to a second position to allow fluid flow between the second syringe and the toxic fluid container such that the second syringe empties a flush solution through the extension set line and into the toxic fluid container.

18. The system of claim 17, wherein the stopcock is turned to a third, closed position prior to disconnecting the extension set.

19. The system of claim 1 1, wherein the cyto admin set further comprises a dedicated universal spike adapter for connection with any spike on any conventional pump set.

20. The system of claim 11, wherein the stopcock includes a stop post that prevents the first and second syringes from being opened at the same time.
